(19) United States
(12) Patent Application Publication
(10) Pub. No.: US 2025/0368588 A1
(43) Pub. Date: Dec. 4, 2025

(54) PROCESS FOR PRODUCING HIGH-PURITY 1-BUTENE AND HIGH-PURITY ISOBUTANE

(71) Applicant: Evonik Oxeno GmbH & Co. KG, Marl (DE)

(72) Inventors: Stephan Peitz, Oer-Erkenschwick (DE); Guido Stochniol, Haltern am See (DE); Vinzenz Fleischer, Marl (DE); Oliver Moeller, Oer-Erkenschwick (DE)

(73) Assignee: Evonik Oxeno GmbH & Co. KG, Marl (DE)

(21) Appl. No.: 19/222,040

(22) Filed: May 29, 2025

(30) Foreign Application Priority Data
Jun. 3, 2024 (EP) 24179589.7

(51) Int. Cl. C07C 5/27

(52) U.S. Cl. CPC C07C 5/27

(57) ABSTRACT

A process produces high-purity 1-butene and high-purity isobutene from two C4 hydrocarbon streams, in which the two C4 hydrocarbon streams are in part processed together. An apparatus carries out the process.

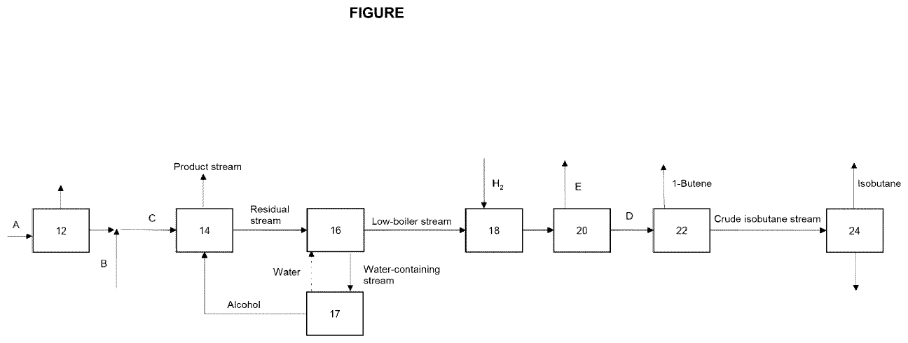

CROSS-REFERENCE TO RELATED APPLICATIONS

This patent application claims priority to European Patent Application No. 24179589.7, filed on Jun. 3, 2024, in the European Patent Office, the entire disclosure of which is hereby incorporated by reference herein.

### BACKGROUND OF THE INVENTION

### Field of the Invention

The present invention relates to a process for producing high-purity 1-butene and high-purity isobutene from two C4 hydrocarbon streams, in which the two streams are in part processed together. The present invention furthermore also relates to an apparatus for carrying out the process according to the invention.

### Description of Related Art

Processes for producing high-purity 1-butene and high-purity isobutane are known and described in the literature, for example in WO 2021/071815 A1. A particular feature of these processes is the fact that isobutene and butadiene are first removed from the C4 hydrocarbon streams used. Isobutene can for example be removed by converting to MTBE or isobutene dimers and then separating off the products formed. Butadiene can be removed by extraction and optionally selective hydrogenation. The further workup is usually effected by distillation, to obtain a high-purity 1-butene stream and a high-purity isobutane stream.

A disadvantage of the process disclosed in WO 2021/071815 A1 is that in the process two separate production lines need to be operated in order to be able to process the two C4 hydrocarbon streams used. However, this is accompanied by high capital and operating costs. In addition, large amounts of energy are needed to operate two separate production lines.

### SUMMARY OF THE INVENTION

The underlying object of the present invention was therefore that of providing a process and an apparatus in which these problems do not occur. The lowest possible number of parallel production plants should be operated in parallel in order to save capital costs and energy. Nevertheless, the process should make it possible to obtain a high-purity 1-butene stream and a high-purity isobutane stream. The savings must therefore not be at the expense of the purity of the 1-butene stream and the isobutane stream.

### BRIEF DESCRIPTION OF THE DRAWINGS

The figure shows a flow diagram of the present invention.

### DETAILED DESCRIPTION OF THE INVENTION

This object was achieved by the present process for producing high-purity 1-butene and high-purity isobutane as per embodiment 1. Preferred configurations are specified in the dependent embodiments.

The object was achieved by the process according to the invention as per embodiment 1. Preferred embodiments are specified in the dependent embodiments. The process according to the invention is a process for producing high-purity 1-butene and high-purity isobutane, wherein the process comprises the following steps of:
a) providing a first C4 hydrocarbon stream A and a second C4 hydrocarbon stream B, with the two streams A and B each containing at least 1,3-butadiene, isobutene, isobutane, 1-butene and 2-butene and with the concentration of isobutane in stream A being higher than in stream B;
b) supplying stream A to an isobutane separation, wherein at least a portion of the isobutane present in stream A is separated off and an isobutane-depleted stream is thus formed;
c) separating off a portion of the isobutane-depleted stream and mixing this portion with stream B to obtain a stream C;
d) supplying stream C and supplying an alcohol, preferably methanol or ethanol, particularly preferably methanol, to a reaction unit, wherein at least a portion of the isobutene present in stream C is converted to ATBE (alkyl tert-butyl ether), preferably MTBE (methyl tert-butyl ether) or ETBE (ethyl tert-butyl ether), particularly preferably MTBE (methyl tert-butyl ether) and/or to isobutene dimers and a reaction output is obtained, wherein the reaction output is subjected to a product separation in which a residual stream, containing at least alcohol, preferably methanol or ethanol, particularly preferably methanol, 1,3-butadiene, 1-butene, 2-butene and isobutane, and a product stream, containing at least the ATBE, preferably MTBE or ETBE, particularly preferably MTBE and/or the isobutene dimers, are obtained;
e) supplying the residual stream to a first separation unit, wherein in the first separation unit a low-boiler stream, containing at least 1,3-butadiene, 1-butene, 2-butene and isobutane, and a water-containing stream, containing at least alcohol, preferably methanol or ethanol, particularly preferably methanol and water, are obtained;
f) supplying the water-containing stream to a recovery unit, in order to at least partially separate the alcohol, preferably methanol and ethanol, particularly preferably methanol, off from the water and recycling at least a portion of the alcohol, preferably methanol or ethanol, particularly preferably methanol, thus obtained to the reaction unit;
g) supplying the low-boiler stream to a hydrogenation, in order to hydrogenate at least a portion of the 1,3-butadiene present, thus obtaining a hydrogenated low-boiler stream;
h) supplying the hydrogenated low-boiler stream to a second separation unit, wherein in the second separation unit a stream D, containing at least isobutane and 1-butene, and a stream E, containing at least n-butane and 2-butene, are obtained;
i) supplying stream D to a third separation unit, wherein in the third separation unit a crude isobutane stream and a stream of high-purity 1-butene are obtained;
j) supplying the crude isobutane stream to a fourth separation unit, wherein in the fourth separation unit an offgas stream and a stream of high-purity isobutane are obtained.

The process according to the invention has the advantage that the two C4 hydrocarbon streams are for the most part treated in one production plant.

The first step a) of the process according to the invention for producing high-purity 1-butene and high-purity isobutane is that of providing a first C4 hydrocarbon stream A and a second C4 hydrocarbon stream B, with the two streams A and B each containing at least 1,3-butadiene, isobutene, isobutane, 1-butene and 2-butene and with the concentration of isobutane in stream A being higher than in stream B.

All typically available C4 hydrocarbon mixtures may be used in the process according to the invention. Suitable C4 hydrocarbon streams are for example light petroleum fractions from refineries, C4 fractions from crackers (for example steam crackers (also: crack C4), hydrocrackers, fluid catcrackers (FCC C4)), mixtures from Fischer-Tropsch syntheses, mixtures from the dehydrogenation of butanes, mixtures from the skeletal isomerization of linear butenes and mixtures formed by metathesis of olefins. These techniques are described in the technical literature.

The C4 hydrocarbon streams A and B used can in principle be produced in the same way or by the same process, but the streams have different amounts of isobutane. However, the process is aimed in particular at the simultaneous production of high-purity 1-butene and isobutane streams from two C4 hydrocarbon streams produced or obtained in different manners. In a particularly preferred embodiment of the present invention, stream A is an FCC C4 stream, i.e. a C4 hydrocarbon stream from a fluid catcracker. Stream B is particularly preferably a crack C4 stream, i.e. a C4 hydrocarbon stream from a steam cracker, or a raffinate I.

While these upstream processes for the production of C4 hydrocarbon streams do produce similar chemical compounds, they result in streams having a different composition of the C4 compounds present. The C4 hydrocarbon streams used in the process according to the invention preferably have the following compositions:

TABLE 1
Typical composition of crack C4, raffinate I and FCC C4

| | Crack C4* | Raffinate I* | FCC C4* |
|---|---|---|---|
| Component | % by mass | % by mass | % by mass |
| Isobutane | 0.6-6 | 2-5 | 20-40 |
| n-Butane | 0.5-11 | 7-12 | 5-15 |
| 1-Butene | 9-25 | 25-31 | 10-20 |
| Isobutene | 10-35 | 40-48 | 10-20 |
| 2-Butene (cis and trans) | 4-20 | 11-15 | 20-35 |
| 1,3-Butadiene | 25-70 | <1 | <1 |
| Further components (e.g. | <3 | <1 | <5 |
| C3 or C5 compounds) | | | |

* = The sum of all components is 100% by mass

The C4 hydrocarbon streams therefore contain different amounts of isobutene depending on the cracking process. Further main constituents are 1,3-butadiene, 1-butene, 2-butene (cis and trans), n-butane and isobutane. Typical isobutene contents in the C4 fraction are 10% to 35% by mass for crack C4 and 10% to 20% by mass for FCC C4.

For the process according to the invention, it is advantageous to for the most part remove polyunsaturated hydrocarbons such as 1,3-butadiene from the feed mixture. This results in a raffinate I. If, therefore, crack C4 is to be used in the process according to the invention, 1,3-butadiene must be at least partially removed before step a). This can be done by known processes, for example by extraction, extractive distillation or complex formation. An alternative to separating off the polyunsaturated hydrocarbons is a selective chemical reaction. For example, 1,3-butadiene can be selectively hydrogenated to linear butenes, as described for example in EP 0 523 482. The 1,3-butadiene can also be at least partially removed by selective reactions of the 1,3-butadiene, for example dimerization to cyclooctadiene, trimerization to cyclododecadiene, or polymerization or telomerization reactions.

### Step b)

The C4 hydrocarbon stream A provided in step a) is supplied in step b) to an isobutane separation, wherein at least a portion of the isobutane present in stream A is separated off and an isobutane-depleted stream is thus formed.

The concentration of the isobutane in the stream A is preferably reduced by means of a distillative step in a distillation to a value of less than 5% by weight. At the same time, the low boilers present in the mixture (for example C3 hydrocarbons, light oxygen-, nitrogen-and sulfur-containing compounds) are also at least partially removed.

In a preferred embodiment, the isobutane-depleted stream from step b) may be supplied to a heavy-boiler separation and/or a separation of nitrogen-containing and/or sulfur-containing and/or oxygen-containing impurities, before the isobutane-depleted stream is sent to step c).

### Heavy-Boiler Separation

The heavy-boiler separation is preferably effected by means of distillation. In the present case, heavy boilers mean, for example, C5 hydrocarbons. Thioethers may also be separated off with the heavy-boiler separation. The thioethers may be formed by the thioetherification of mercaptans. The thioetherification is used to remove the mercaptans. Such a process is disclosed for example in WO 2014/009148 A1.

The distillative separation of the heavy boilers such as C5 hydrocarbons and possibly thioethers is effected in at least one distillation column. The heavy boilers accumulate in the bottom. The isobutane-depleted stream accordingly accumulates at the top of the distillation column.

A distillation column preferably used in this process step preferably has 40 to 150 theoretical plates, with preference 40 to 100 and particularly preferably 50 to 80 theoretical plates.

The reflux ratio, depending on the number of plates implemented, the composition of the column feed and the required purities of the distillate and the bottom product, is preferably between 0.5 and 5, particularly preferably between 1 and 2.5. The reflux ratio is defined here as the mass flow rate of the reflux divided by the mass flow rate of the distillate. The column is preferably operated at an operating pressure of 0.1 to 2.0 MPa (absolute), with preference of 0.5 to 1.2 MPa (absolute).

The column may be heated using steam, for example. Depending on the chosen operating pressure, the condensation may be effected against cooling brine, cooling water or air. However, the top vapours from the column may also be thermally integrated with other columns in the process, for example with the column for separating off the isobutane. In this case, the condenser of the column serves simultaneously as the evaporator of the low-boiler column. The bottom product can be utilized thermally or used as a starting material for other processes, for example in a synthesis gas plant.

Various processes can be used for separating off nitrogen-containing and/or sulfur-containing and/or oxygen-containing impurities.

Water Washing Water washing can be used to fully or partially remove hydrophilic components from the isobutane-depleted stream, for example nitrogen components. Examples of nitrogen components are acetonitrile or N-methylpyrrolidone (which may, for example, originate from a 1,3 butadiene extractive distillation). Oxygen compounds (for example acetone from an FCC unit) can also be partially removed by water washing. The isobutane-depleted stream is saturated with water after water washing. In order to avoid having two phases in the downstream process steps in the reactor, the reaction temperature should be about 10° C. higher than the temperature of the water washing.

### Adsorbent

Adsorbents are used to remove impurities from the isobutane-depleted stream. This can be advantageous, for example, if noble metal catalysts are used in one of the process steps. Nitrogen or sulfur compounds are often removed via upstream adsorbers. Examples of adsorbents are aluminium oxides, molecular sieves, zeolites, activated carbon, metal-impregnated clays. Adsorbents are sold by various companies, for example by Alcoa (Selexsorb®).

### Drying

Water which may be present in the isobutane-depleted stream, and which may, for example, originate from the water washing, can be removed by known drying methods. Examples of suitable processes include the distillative separation of the water as an azeotrope. An azeotrope with C4 hydrocarbons present can often be utilized or entraining agents can be added.

### Step c)

In step c), a portion of the isobutane-depleted stream obtained from step b) is separated off. The isobutane-depleted stream is consequently separated. The separation at least of a portion of the isobutane-depleted stream/separation of the stream in step c) may for example be effected via a valve with flow control. Appropriate internals and structures are familiar to those skilled in the art.

The portion of the isobutane-depleted stream separated off is then mixed with stream B to obtain a stream C, which is sent to step c) for further processing.

The other portion of the isobutane-depleted stream is processed independently of stream C and may, for example, be supplied to a separate isobutene conversion for the formation of ATBE, preferably for the formation of MTBE or for the formation of ETBE, or for the formation of isobutene dimers.

### Step d)

After mixing the two streams to give stream C in step c), stream C and an alcohol, preferably methanol or ethanol, particularly preferably methanol, are sent to a reaction unit, wherein at least a portion of the isobutene present in stream C is converted to ATBE (alkyl tert-butyl ether), preferably MTBE (methyl tert-butyl ether) or ETBE (ethyl tert-butyl ether), particularly preferably MTBE (methyl tert-butyl ether) and/or to isobutene dimers and a reaction output is obtained. The reaction output is subjected to a product separation in which a residual stream, containing at least alcohol, preferably methanol or ethanol, particularly preferably methanol, 1,3-butadiene, 1-butene, 2-butene and isobutane, and a product stream, containing at least the ATBE, preferably MTBE or ETBE, particularly preferably MTBE and/or the isobutene dimers, are obtained.

The conversion in step d) is effected in one or more reactors that are suitable for the respective conversion. If there are two or more reactors, these reactors can be connected in parallel or in series. A variety of configurations are therefore possible. For example, the conversion may be processed in a series of reactors in a fixed bed reactor or a series of fixed bed reactors and may include an intermediate separation stage in order to remove a portion of the product (ATBE or isobutene dimer). In some embodiments, an upstream reactor output may be supplied to a final reactor, which may be a reactive distillation, in which a simultaneous reaction of at least a portion of the remaining isobutene and a separation of dimer or ATBE from the remaining C4 components, including n-butane, isobutane, 1-butene and 2-butene, is enabled. The reactive distillation then also corresponds to the product separation.

In a preferred embodiment of the present invention, the conversion in step d) is carried out in at least two reaction stages, with at least the last reaction stage being carried out as a reactive distillation. The ATBE conversion in this step is preferably above 70%, particularly preferably above 90%.

The alcohol used in the conversion in step d), preferably methanol or ethanol, particularly preferably methanol, can act either as a reactant (for the production of ATBE or MTBE or ETBE) or as a moderator (for the selective dimerization to isobutene dimers). The two alternatives are explained in more detail below.

### Production of ATBE, Preferably MTBE or ETBE

If ATBE or MTBE or ETBE is produced in step d), the production of ATBE is preferably carried out in two stages. The first stage of the ATBE synthesis in the process step d) according to the invention is preferably carried out in fixed bed reactors, the second stage of the conversion is preferably effected in a reactive distillation. In this context, the reactive distillation simultaneously represents the product separation. The first stage of the ATBE synthesis is preferably carried out in at least two, particularly preferably three, fixed bed reactors. As reactors in which the alcohol, preferably methanol or ethanol, particularly preferably methanol, is reacted with the isobutene to close to the thermodynamic equilibrium, conventional fixed bed reactors (tube bundle reactors, adiabatic fixed bed reactors, recycle reactors) may be used. As a result of the two-stage ATBE synthesis, isobutene residual concentrations in the reaction output in particular of less than 1000 ppm by mass, preferably 800 ppm by mass and particularly preferably less than 500 ppm by mass, based on the C4 mixture in the distillate, can be obtained.

In the first stage, the isobutene is preferably converted until the thermodynamic equilibrium of ATBE, alcohol, preferably methanol or ethanol, particularly preferably methanol and isobutene, is established, with an isobutene conversion preferably of greater than 94%, particularly preferably greater than 96%, being achieved. The reactors of the first stage are preferably operated at a temperature of 20 to 110° C., preferably 25 to 70° C. and a pressure of 0.5 to 5 MPa, preferably 0.7 to 2 MPa.

Since the thermodynamic equilibrium between alcohol/isobutene and ether at low temperature lies predominantly on the side of the ether, it is preferable to operate the first of the reactors for the purpose of a high reaction rate at a higher temperature than the following reactors in which the equilibrium position is exploited.

The molar ratio of alcohol to isobutene (alcohol:isobutene) in the feed to the first reactor of the first stage is preferably in the range from 10:1 to 1:1, particularly preferably from 5:1 to 1.1:1, and very particularly preferably in the range from 1.8:1 to 1.2:1.

The second stage of the ATBE synthesis is preferably carried out in a reactive distillation column. In addition to the further conversion of isobutene to ATBE, also effected in the reactive distillation is the product separation into the residual stream, containing at least alcohol, preferably methanol or ethanol, particularly preferably methanol, 1,3-butadiene, 1-butene, 2-butene and isobutane, and the product stream, containing at least the ATBE and/or the isobutene dimers. The residual stream thus separated off is further treated in step e) according to the invention.

The second stage of the ATBE synthesis is further preferably carried out in a reactive distillation column, which is operated in a pressure range with a positive pressure of 0.5 to 1.5 MPa, preferably 0.75 to 1.0 MPa and at a temperature in the reaction zone of 50° C. to 90° C., preferably 55 to 70° C., at a reflux ratio between 0.5 and 1.5, preferably between 0.7 and 0.9, on an acidic ion exchange resin. The reflux ratio by definition denotes the ratio of the reflux stream into the column to the discharged distillate stream. The temperature of the column feed, irrespective of its composition, the reaction pressure in the column and the throughput, is preferably between 50° C. and 90° C., preferably between 60° C. and 75° C.

The feed to the reactive distillation column may be effected above or below, preferably below, the catalyst zone. The feed to the reactive distillation column is preferably effected below the reactive packing, preferably 3 to 13, particularly preferably 4 to 10, theoretical plates below the reactive packing.

Optionally, additional alcohol, preferably methanol or ethanol, particularly preferably methanol, may be fed into the second stage. This can be effected together with the feed from the first stage or else also at one or more points in the reactive distillation column, for example at the column top and/or on, between and/or beneath the catalyst bed.

The reactive distillation column preferably contains the catalyst in the rectifying column and below and above the catalyst packing are preferably located separation trays or distillation packings. The catalyst can either be integrated into a packing, such as for example in KataMax® packings, KataPak® packings or MultiPak® packings, or polymerized onto shaped bodies. Preference is given to using KataMax® packings.

Preferably, the reactive distillation column has, above the catalyst packing, a region of pure distillative separation. Preferably, the zone above the catalyst packing has 5 to 20, in particular 10 to 15, separation stages. The separation zone below the catalyst comprises 12 to 36, in particular 20 to 30, separation stages. The catalyst zone can be estimated with a distillative effect of 1 to 5 theoretical plates per metre of packing height. The height of the catalyst zone/reactive zone can be determined by simple preliminary tests depending on the desired isobutene conversion. The catalyst amount is preferably selected to be large enough that an isobutene conversion of 75% to 99%, preferably from 85% to 98% and particularly preferably from 95% to 97%, based on the isobutene content in the feed to the reactive distillation is achieved.

As catalysts, solid acidic ion exchange resins containing sulfonic acid groups are preferably used in the ATBE synthesis in step d). Suitable ion exchange resins are for example those produced by sulfonation of phenol/aldehyde condensates or of co-oligomers of aromatic vinyl compounds. Examples of aromatic vinyl compounds for the production of the co-oligomers are: styrene, vinyltoluene, vinylnaphthalene, vinylethylbenzene, methylstyrene, vinylchlorobenzene, vinylxylene and divinylbenzene. Co-oligomers formed by reaction of styrene with divinylbenzene are used in particular as precursors for the production of ion exchange resins having sulfonic acid groups. The resins can be produced in gel-like, macroporous or sponge-like form. The properties of these resins, in particular specific surface area, porosity, stability, swelling/shrinkage and exchange capacity, can be varied via the production process.

In the process according to the invention, the ion exchange resins may be used in their H form. Strongly acidic resins of styrene-divinylbenzene type are sold inter alia under the following trade names: Duolite® C20, Duolite® C26, Amberlyst@ 15, Amberlyst@ 35, Amberlite® IR-120, Amberlite® 200, Dowex® 50, Lewatit® SPC 118, Lewatit® SPC 108, K2611, K2621, OC 1501. As ion exchange resins, preference is given to using the types Amberlyst® 15, Amberlyst® 35 or Lewatit® K2621.

The ATBE obtained as bottom product in the second stage of the ATBE synthesis, preferably the reactive distillation column, can be used for various purposes. Since it contains only extremely small amounts of alkyl sec-butyl ether (ASBE), it is suitable for the production of high-purity isobutene via its retrocleavage, since virtually no linear butenes can be formed by retrocleavage of the alkyl sec-butyl ether. Due to the low content of by-products (ASBE and C8 olefins), the ATBE obtained in this way can, after separation off from the remaining alcohols, be used as a solvent in analysis or in organic syntheses. It may also be used as a component for petrols.

Preferably, the ATBE synthesis in process step d) of the process according to the invention is carried out such that in the second stage an overhead product, containing alcohol, preferably methanol or ethanol, particularly preferably methanol and a C4 hydrocarbon mixture (1,3-butadiene, 1-butene, 2-butene and isobutane) having an isobutene content of less than 1000 ppm by mass, based on the C4 hydrocarbon mixture, and a product stream as bottom product, containing ATBE, are obtained.

### Production of Isobutene Dimers

In an alternative, the isobutene is converted to isobutene dimers (diisobutene) in step d) of the process according to the invention. The production of the isobutene dimers in step d) from the isobutene can in principle be homogeneously catalysed, i.e. using catalysts soluble in the reaction mixture, or heterogeneously catalysed, i.e. using catalysts insoluble in the reaction mixture. The production of the isobutene dimers in step d) is preferably effected over solid heterogeneous catalysts, which are further preferably arranged in the fixed bed, such that laborious catalyst separation is dispensed with.

As solid catalysts, it is possible to use acidic substances that are insoluble in the reactant/product mixture. Most of these catalysts belong to one of the following groups:
a) mineral acids (e.g. sulfuric acid or phosphoric acid) on a support material (e.g. aluminium oxide or silicon dioxide),
b) zeolites or other aluminosilicates with or without doping with further metals, in particular with transition metals or
c) acidic ion exchange resins, especially acidic cation exchangers.

Due to the higher selectivity for the formation of isobutene oligomers and due to the lower formation of by-products, acidic ion exchange resins are preferably used as catalyst. Suitable ion exchange resins are for example those produced by sulfonation of phenol/aldehyde condensates or of co-oligomers of aromatic vinyl compounds. Examples of aromatic vinyl compounds for the production of the co-oligomers are: styrene, vinyltoluene, vinylnaphthalene, vinylethylbenzene, methylstyrene, vinylchlorobenzene, vinylxylene and divinylbenzene. Co-oligomers formed by reaction of styrene with divinylbenzene are used in particular as precursors for the production of ion exchange resins having sulfone groups. The properties of these resins, in particular specific surface area, porosity, stability, swelling/shrinkage and exchange capacity, can be varied via the production process. The resins can be produced in gel-like, macroporous or sponge-like form. Strongly acidic resins of styrene-divinylbenzene type are sold inter alia under the following trade names: CT 151 from Purolite®, Amberlyst@ 15, Amberlyst@ 35, Amberlite® IR-120, Amberlite® 200, Dowex® M-31, K 2611, K 2431.

The acidic ion exchange resin is expediently adjusted to an activity that enables the oligomerization of the isobutene, but hardly catalyses the co-oligomerization of isobutene with linear butenes, the oligomerization of the linear butenes and the isomerization of the linear butenes. In addition, the evolution of heat in the reactor is thereby adjusted to a technically readily manageable value.

The desired catalyst activity can be adjusted with the aid of moderators. These substances are passed together with the reactant over the catalyst. Used as moderator is alcohol, preferably methanol or ethanol, particularly preferably ethanol, as a pure substance or as a mixture. The production of the isobutene dimers is therefore preferably carried out in the presence of these moderators. When using moderators, preference is given to setting molar ratios of 0.01 to 5, preferably 0.01 to 1, in particular 0.01 to 0.7, mol of moderators per mole of isobutene.

A reactor in the process according to the invention may contain a mixture of ion exchange resins of different reactivities. It is likewise possible for a reactor to contain catalysts having different activities, for example arranged in layers. If more than one reactor is used, the individual reactors may be filled with catalysts of identical or different activities.

The reactors used in the industrial process may be operated adiabatically, polytropically or practically isothermally. Practically isothermally means that the temperature at any point in the reactor is not more than 10° C. higher than the temperature at the reactor entrance. In the case of adiabatic operation of the reactors, it is usually expedient to connect two or more reactors in series and preferably to effect cooling between the reactors. Reactors that are suitable for polytropic or practically isothermal operation are for example tube bundle reactors, water-cooled tubular reactors (cooling system on the jacket side), stirred tanks and loop reactors. It is possible to combine two or more reactors, and also different designs. It is also possible to operate reactors with recycling of product. In a preferred embodiment of the present invention, the production of isobutene dimers is carried out in at least two series-connected reactors, wherein there is an intermediate separation of the dimers between the reactors.

The temperatures in the production of the isobutene dimers in step d) are preferably in the range from 15 to 160° C., preferably in the range from 40 to 110° C.

The conversion may be effected with or without the addition of an additional solvent. As solvent, preference is given to using saturated hydrocarbons, in particular C4, C8 or C12 hydrocarbons. When solvents are added, their proportion is 0% to 60% by mass, preferably 0% to 30% by mass.

The conversion according to the invention may be carried out at a pressure equal to or above the vapour pressure of stream C at the respective reaction temperature, preferably at a pressure of below 40 bar, i.e. stream C would be present wholly or partially in liquid phase during the dimerization. If the reaction is to be carried out completely in the liquid phase, the pressure should preferably be 2 to 4 bar higher than the vapour pressure of the reaction mixture, in order to avoid evaporation problems in the reactors.

Even if the reaction is operated at a pressure at which the reaction mixture is not completely liquid (for example, in a reactive distillation), the oligomerization according to the process of the invention still takes place in the liquid phase, that is to say on a “moist”, i.e. liquid-wetted, catalyst.

The total conversion of isobutene to dimers can be adjusted via the type and amount of the catalyst used, the reaction conditions set and the number of reactors. In the process according to the invention, with preference 30% to 95%, preferably 50% to 80%, particularly preferably 55% to 70%, of the isobutene present in the reactant is converted.

The reaction mixture from the dimerization can be worked up in various ways. The product separation is preferably effected by distillation. The use of a reactive distillation is also possible. The product separation results in a residual stream containing at least methanol, 1,3-butadiene, 1-butene, 2-butene and isobutane, and a product stream containing at least the isobutene dimers.

The distillation is preferably operated at a pressure of 1 to 10 bara (bara =bar absolute), particularly preferably at a pressure of 4 to 7 bara. The temperatures in the bottom are preferably from 120 to 220° C., particularly preferably from 170 to 200° C. The reflux ratio is preferably set to values of 0.1 to 1.5, preferably of 0.3 to 1.0. The distillation is preferably carried out in a column with a number of trays in the range from 20 to 40, preferably in the range from 25 to 35. The residual stream thus separated off is further treated in step e) according to the invention.

The product stream separated off mainly contains isobutene dimers (C8 hydrocarbons) and may possibly include a portion of the moderator used. In addition to the diisobutene, it may also contain co-dimers and higher oligomers (C12, C16+, etc.). The proportion of co-oligomers is preferably below 25% by mass. The product stream may be separated in further distillation steps. For example, it is possible to separate off a fraction comprising high-purity diisobutene in order to use it separately, for example for chemical syntheses. For use as a fuel component for petrol engines, it may be necessary to separate off high-boiling components (preferably boiling point >220° C.).

It is also possible to fully or partially hydrogenate the oligomers/dimers. Methods for hydrogenating the products of the oligomerization to give the corresponding paraffins are sufficiently well known to those skilled in the art. In a preferred embodiment, the hydrogenation is carried out in liquid phase over a solid catalyst that is not soluble in the hydrogenation material. As hydrogenation catalysts, preference is given to using supported catalysts consisting of an inorganic support and containing as active metal platinum and/or palladium and/or nickel. The temperature at which the hydrogenation is carried out is preferably in the range from 10 to 250° C. and the pressure is between 1 and 100 bar.

After the hydrogenation, further fractions may be obtained by distillative separation. Fuel additives having certain properties are obtainable from these and from the unhydrogenated fractions by blending. In addition, some fractions may be used as solvents.

### Step e)

In the subsequent step e), the residual stream from step d) is sent to a first separation unit, wherein in the first separation unit a low-boiler stream, containing at least 1,3-butadiene, 1-butene, 2-butene and isobutane, and a water-containing stream, containing at least the alcohol, preferably methanol or ethanol, particularly preferably the methanol and water, are obtained. Step e) therefore relates in particular to the separation of the alcohol, preferably of methanol or ethanol, particularly preferably of methanol, off from the C4 hydrocarbons.

The separation of the alcohol, preferably of the methanol or ethanol, particularly preferably of the methanol, off from the residual stream is effected in particular by extraction with water or an aqueous solution as washing medium. The alcohol, preferably the methanol or ethanol, is therefore preferably washed out from the residual stream in an extraction step with water or an aqueous solution. Preferably, an aqueous solution with a pH of greater than or equal to 8, preferably of 8 to 12, is used. The pH can be adjusted, for example, by adding sodium hydroxide solution and/or sulfuric acid. This extraction by the known standard processes of the art can for example be effected in an extraction column or in a cascade of mixers and separating vessels. It has various advantages over other processes, by way of example low investment and low operating costs.

The separation results in a water-containing stream, containing at least the alcohol, preferably methanol or ethanol, particularly preferably methanol and water, and a low-boiler stream, containing at least 1,3-butadiene, 1-butene, 2-butene and isobutane. The residual content of alcohol in the low-boiler stream is preferably less than 0.2% by mass, particularly preferably less than 500 ppm by mass, very particularly preferably less than 50 ppm by mass.

The first separation unit for the extraction of the alcohol, preferably of the methanol or ethanol, particularly preferably of the methanol, preferably comprises at least one extraction column. The at least one extraction column preferably has from 2 to 25, particularly preferably from 5 to 15, theoretical plates and is preferably operated at temperatures of 10 to 90° C. and pressures of at least 0.1 MPa above the vapour pressure of the C4 hydrocarbons. The mass ratio of the washing medium to the residual stream supplied is preferably from 1:5 to 1:40.

Preferably, the residual stream is transferred to an extraction column, into which it is fed in countercurrent with the extractant via a feed located at the top. The laden extractant is the water-containing stream and can be withdrawn via the discharge at the bottom of the extraction column.

The alcohol-laden washing water from the extraction, that is to say the water-containing stream, is worked up in step f) and then at least partially recycled to the extraction.

### Step f)

In step f) the water-containing stream is sent to a recovery unit, in order to at least partially separate the alcohol, preferably the methanol or ethanol, particularly preferably the methanol, off from the water. At least a portion of the alcohol, preferably of the methanol or ethanol, particularly preferably of the methanol, thus obtained is recycled to the reaction unit in step d).

The water-containing stream in the recovery unit can be worked up, for example, by a distillation, in which a virtually alcohol-free water fraction is obtained in the bottom and methanol is obtained as overhead product. The distillation is preferably carried out at a positive pressure, for example in the range from 1.1 to 1.5 barg. The temperature in the bottom is preferably 110 to 140° C. The temperature at the top is 80 to 95° C. The separation of alcohol, preferably methanol or ethanol and water is known in principle to those skilled in the art. The alcohol, preferably the methanol or ethanol, can be returned to the ATBE synthesis or the production of the isobutene dimers in step d). The water fraction from the bottom can be recycled to the first separation unit in step e) for reuse.

### Step g)

The low-boiler stream is sent in step g) to a hydrogenation, in order to hydrogenate at least a portion of the 1,3-butadiene present, thus obtaining a hydrogenated low-boiler stream. If traces of butadiene have not already been removed prior to process step g), they can thus be removed from the residual stream by selective hydrogenation (SHP).

The hydrogenation in step g) can be effected in liquid phase over a palladium-containing fixed bed catalyst with hydrogen with the addition of carbon monoxide as a moderator. Hydrogen and carbon monoxide are here completely dissolved in the hydrocarbon mixture. The amount of hydrogen added is at least that which is stoichiometrically necessary for the hydrogenation of the polyunsaturated compounds to give the simple olefins. It can be calculated from the composition of the low-boiler stream to be hydrogenated.

The amount of CO to be based on the mass of the low-boiler stream is from at least 0.05 ppm by mass to 100 ppm by mass. Amounts above 20 ppm do not by default lead to any further improvement in the hydrogenation results, and so amounts from 0.05 to 10 ppm by mass are preferred. The optimal amount of CO to be metered in in the respective process can be easily determined experimentally, as described in DE 31 43 647.

The catalyst includes 0.1% to 2% by mass of palladium and/or platinum on a support. Such supports include, for example, aluminium oxide, silica gel, aluminosilicate and activated carbon. An amount of hydrocarbons in the range from 5 to 300 litres is preferably put through per litre of catalyst used.

The temperature at which the hydrogenation is carried out is from 0 to 75° C. In order to avoid any free water, the hydrogenation is expediently operated at a higher temperature than the extraction in process step e).

The process pressure must be sufficiently high to maintain the liquid phase at the chosen temperature and to bring a sufficient amount of hydrogen and carbon monoxide into solution. The reaction pressure is less than 20 MPa, with preference less than 6 MPa, preferably less than 2 MPa. A typical reaction pressure is 1.5 MPa.

The hydrogenation may be carried out in a single-or multistage manner. Single-stage means that only a single reactor is used. Multistage accordingly means that there are two or more reactors. Single-stage execution is preferred for cost reasons. Alternatively, the hydrogenation is carried out in multiple stages, preferably in two stages. Hydrogen is fed in upstream of each of the reactors, and carbon monoxide is preferably fed into the first of the reactors. The reactors may be operated with product recycling. In a preferred embodiment, the hydrogenation of the at least a portion of the 1,3-butadiene is carried out in at least two reaction stages, with at least the last reaction stage being carried out in the presence of 0.05 to 100 ppm by mass of CO.

### Step h)

The hydrogenated low-boiler stream obtained from step g) contains at least n-butane, isobutane, 1-butene and 2-butene (cis and trans), but only small amounts-if any-in the ppm range of 1,3-butadiene and/or isobutene. The hydrogenated low-boiler stream is then sent in step h) to a second separation unit, wherein in the second separation unit a stream D, containing at least isobutane and 1-butene, and a stream E, containing at least n-butane and 2-butene, are obtained. Separation into streams D and E is preferably effected by distillation.

Isobutane and 1-butene can be completely or partially separated by distillation, as stream D, off from the hydrogenated low-boiler stream, also referred to in the literature as raffinate II. The stream E additionally obtained, also referred to as raffinate III, typically contains mainly 2-butenes, n-butane and possibly a proportion of 1-butene. Stream E is discharged from the present process and can be used, for example, as a feedstock mixture for an oligomerization. The distillative separation can be effected in apparatuses typically used for the separation of such hydrocarbon mixtures, for example distillation or fractionation columns.

In a preferred embodiment, the distillative separation is carried out in a superfractionation column. The feed to this superfractionation column is preferably effected in the lower half, preferably in the lower third of the superfractionation column. Because of the narrow boiling point of the mixture to be separated, process step h) is preferably carried out in a superfractionation column which has more than 100, preferably more than 125, particularly preferably more than 150 theoretical plates, and very particularly preferably 150 to 200 theoretical plates. The superfractionation column may be designed as a packed column or tray column. It is preferably designed as a tray column.

The reflux ratio (reflux amount to distillate takeoff) in the superfractionation column is, depending on the number of plates implemented and on the operating pressure, with preference less than or equal to 20, preferably less than 14, particularly preferably less than 11. The condensation may be carried out against cooling water or air. It would be possible to render the condensation energy utilizable, for example by means of vapour compression or a heat pump. The distillate vessel is preferably designed as a liquid-liquid separator. As a result, any water present in the feed stream can be separated off as a second phase in the distillate vessel and a technically anhydrous bottom product (stream E) can be obtained.

The separation according to process step h) is preferably carried out at a pressure of 0.4 to 1.0 MPa absolute, preferably at a pressure of 0.5 to 0.7 MPa absolute. The temperature at which the separation is carried out is with preference 35 to 80° C., preferably 40 to 65° C.

Columns for the separation of substance mixtures typically have at least one evaporator, via which the energy necessary for accomplishing the separation task is introduced. For heating the evaporator of the superfractionation column used in process step h), a customary heat transfer medium, such as for example steam or hot water, and preferably waste heat from other processes can be used. In the latter case, it may be advantageous to equip the column with more than one evaporator.

The superfractionation column is preferably equipped as a simple column with at least one evaporator and at least one condenser. Due to the high energy requirement and the small temperature difference between the bottom and the top of the column, energy-saving circuits are particularly preferred embodiments. Reference is made here, by way of example, to the vapour compression method. A further particularly preferred circuit is the dual-pressure circuit (double-effect distillation) in integration with a second column. Here, one of the columns is operated at such a high pressure that its condensation temperature is sufficient to heat the other column. In the thermal interconnection of columns with different separation tasks, any suitable column from the process according to the invention, but also a column that is present outside of the process according to the invention at the plant site, may in principle be interconnected with the column of process step h). Particularly preferably, the second column is the at least one column from the following process step i). It is further preferable here if the superfractionation column from the present step h) has a higher pressure, since the simpler separation task is accomplished here.

### Step i)

Stream D is sent in the following step i) to a third separation unit, wherein in the third separation unit a crude isobutane stream and a stream of high-purity 1-butene are obtained. In this step, the 1-butene is separated from the isobutane in stream D. The separation is preferably effected by means of distillation.

The separation in step i) results in a high-purity 1-butene stream having a proportion of 1-butene of at least 99% by mass based on the overall 1-butene stream.

In a preferred embodiment, the 1-butene stream has a purity of over 99.2% by mass, further preferably over 99.3% by mass, further preferably over 99.4% by mass and particularly preferably over 99.5% by mass, based in each case on the overall 1-butene stream. Further preferably, the 1-butene stream with preference contains less than 5000 ppm by mass, preferably less than 2000 ppm by mass, and particularly preferably less than 1500 ppm by mass, of isobutene.

In a preferred embodiment, the separation of the 1-butene is effected in at least one distillation column in which very pure 1-butene is obtained as bottom product. The overhead product obtained is a crude isobutane stream, which may contain low boilers (for example C3 hydrocarbons) and is sent to step j).

Preferably, the separation in step i) is carried out in a superfractionation column. The feed to this superfractionation column is preferably effected in the upper half, preferably in the lower half of the upper half of the column. Because of the narrow boiling point of the mixture to be separated, the superfractionation column is designed with preference with more than 100, preferably more than 125, particularly preferably more than 150, and very particularly preferably from 150 to 200 theoretical plates. The superfractionation column is designed with preference as a packed column or tray column, preferably as a tray column. The reflux ratio (reflux amount to distillate takeoff) is, depending on the number of plates implemented and on the operating pressure, with preference less than or equal to 100, preferably less than 70, particularly preferably less than 60. The reflux ratio is very particularly preferably from 30 to 60. The condensation may be carried out against cooling water or air. The distillate vessel is preferably designed as a liquid-liquid separator. As a result, any water present in the feed stream can be separated off as a second phase in the distillate vessel and a technically anhydrous bottom product can be obtained.

Columns for the separation of substance mixtures typically have at least one evaporator, via which the energy necessary for accomplishing the separation task is introduced. For heating the evaporator of the column, a customary heat transfer medium, such as for example steam or hot water, and preferably waste heat from other processes can be used. In the latter case, it may be advantageous to equip the column with more than one evaporator.

The superfractionation column is preferably equipped as a simple column with at least one evaporator and at least one condenser. Due to the high energy requirement and the small temperature difference between the bottom and the top of the column, energy-saving circuits are particularly preferred embodiments. Reference is made here, by way of example, to the vapour compression method. A further particularly preferred circuit is the dual-pressure circuit (double-effect distillation) in integration with a second column. Here, one of the columns is operated at such a high pressure that its condensation temperature is sufficient to heat the other column. In the thermal interconnection of columns with different separation tasks, any suitable column from the process according to the invention, but also a column that is present outside of the process according to the invention at the plant site, may in principle be interconnected with the column according to the invention of process step f). Particularly preferably, the superfractionation column in this step i) is the second column and the column of step h) is the first column. Here, one of the columns, preferably the first column, is operated at such a high pressure that its condensation temperature is sufficient to heat the other column.

In addition to this preferred embodiment of the process step i) according to the invention, it is also possible to separate firstly any low boilers present off from stream D in a first distillation column as overhead product, wherein a mixture containing mainly 1-butene and isobutane is obtained in the bottom of the column. In a second column, which may be designed like the superfractionation column described above, this bottom mixture can be separated into 1-butene, which is obtained as bottom product, and an isobutane-rich fraction (overhead product).

High-purity 1-butene produced by the process according to the invention is a sought-after intermediate. It can be used, for example, as a comonomer in the production of polyethylene (LLDPE or HDPE) and also of ethylene-propylene copolymers. It is further used as an alkylating agent and is a starting material for the production of butan-2-ol, butene oxide, valeraldehyde. A further use for the virtually isobutene-free 1-butene produced according to the invention is the production of n-butene oligomers, in particular by the Octol process.

In an alternative embodiment, a hydrogenation may be effected after step i) in order to hydrogenate traces of olefins to the corresponding alkanes. This hydrogenation of traces of olefins is known to those skilled in the art.

### Step j)

The crude isobutane stream separated off from step i) is sent to a fourth separation unit, wherein in the fourth separation unit an offgas stream and a stream of high-purity isobutane are obtained.

The isobutane obtained in the workup with preference has a purity of at least 95% by mass of isobutane, further preferably a purity of over 95.2% by mass, further preferably a purity of over 95.3% by mass, further preferably a purity of over 95.4% by mass and particularly preferably a purity of over 95.5% by mass, coupled with a total butane content (n-butane +isobutane) of at least 99.5% by mass, preferably 99.6% by mass, particularly preferably 99.7% by mass. The high-purity isobutane stream further preferably contains less than 1000 ppm by mass, particularly preferably less than 200 ppm by mass, of olefins. The high-purity isobutane stream further preferably contains less than 100 ppm by mass, particularly preferably less than 10 ppm by mass, of oxygenates such as for example dimethyl ether or methanol.

The distillation is preferably carried out at a positive pressure, preferably in the range from 8 to 12 barg. The temperature in the bottom of the distillation column is preferably 65 to 85° C. At the top of the distillation column, the temperature is preferably in the range from 60 to 80° C.

A further aspect of the present invention is the provision of an apparatus for carrying out a process according to the invention for producing high-purity 1-butene and high-purity isobutane, comprising an isobutane separation unit (12) comprising at least one distillation column in which at least a portion of the isobutane present in stream A is separated off; a reaction unit (14) comprising at least one reactor and in which at least a portion of the isobutene present in stream C is converted to MTBE and/or to isobutene dimers; a first separation unit (16) comprising at least one distillation column and in which a low-boiler stream, containing at least 1,3-butadiene, 1-butene, 2-butene and isobutane, is separated off; a recovery unit (17) for recovering methanol; a hydrogenation unit (18) comprising at least one hydrogenation reactor and in which at least a portion of the 1,3-butadiene present in the low-boiler stream is selectively hydrogenated; a second separation unit (20) comprising at least one distillation column and in which a stream D, containing at least isobutane and 1-butene, is separated off; a third separation unit (22) comprising at least one distillation column and in which high-purity 1-butene is separated off; and a fourth separation unit (24) comprising at least one distillation column and in which high-purity isobutane is separated off.

The present invention is described with reference to the flow diagram in the figure. However, the representation shown in this figure should be understood as explanatory only and not as limiting.

The figure shows a flow diagram of the present invention. The first C4 hydrocarbon stream (A) is sent to an isobutane separation (12), wherein at least a portion of the isobutane present in stream A is separated off and an isobutane-depleted stream is thus formed. The isobutane-depleted stream is mixed with the second C4 hydrocarbon stream (B) to obtain a stream (C) and passed to a reaction unit (14). The isobutene present is at least partially reacted with an alcohol, preferably with methanol or ethanol, particularly preferably with methanol to give ATBE (alkyl tert-butyl ether), preferably MTBE (methyl tert-butyl ether) or ETBE (ethyl tert-butyl ether), particularly preferably MTBE (methyl tert-butyl ether) and/or to give isobutene dimers. The reaction unit (14) also comprises a product separation (not explicitly shown). The reaction output is subjected to a product separation in which a residual stream, containing at least alcohol, preferably methanol or ethanol, particularly preferably methanol, 1,3-butadiene, 1-butene, 2-butene and isobutane, and a product stream, containing at least the MTBE and/or the isobutene dimers, are obtained. The product stream is discharged and the residual stream is sent to a first separation unit (16), in which a low-boiler stream, containing at least 1,3-butadiene, 1-butene, 2-butene and isobutane, and a water-containing stream, containing at least alcohol, preferably methanol or ethanol, particularly preferably methanol and water, are obtained. In the recovery unit (17), the alcohol, preferably methanol and ethanol, particularly preferably methanol, is at least partially separated off from the water and at least a portion of the alcohol, preferably methanol or ethanol, particularly preferably methanol, thus obtained is recycled to the reaction unit (14). The water may be recycled to the first separation unit (16), indicated by the dashed line. The low-boiler stream from the first separation unit (16) is sent to a hydrogenation (18) in which 1,3-butadiene is selectively hydrogenated and a hydrogenated low-boiler stream is obtained. The hydrogenated low-boiler stream is then sent to a second separation unit (20), in which a stream D, containing at least isobutane and 1-butene, and a stream E, containing at least n-butane and 2-butene, are obtained. Stream E is discharged and stream D is sent to a third separation unit (22), in which a crude isobutane stream and a stream of high-purity 1-butene are obtained. The crude isobutane stream is worked up in a fourth separation unit (24), to obtain an offgas stream and a stream of high-purity isobutane.

1. A process for producing high-purity 1-butene and high-purity isobutane, the process comprising:
a) providing a first C4 hydrocarbon stream A and a second C4 hydrocarbon stream B, with the first C4 hydrocarbon stream A and the second C4 hydrocarbon stream B each containing at least 1,3-butadiene, isobutene, isobutane, 1-butene and 2-butene and with a concentration of isobutane in the first C4 hydrocarbon stream A being higher than in the second C4 hydrocarbon stream B;
b) supplying the first C4 hydrocarbon stream A to an isobutane separation, wherein at least a portion of the isobutane present in the first C4 hydrocarbon stream A is separated off and an isobutane-depleted stream is thus formed;
c) separating off a portion of the isobutane-depleted stream and mixing this portion with the second C4 hydrocarbon stream B to obtain a stream C;
d) supplying stream C and supplying an alcohol to a reaction unit, wherein at least a portion of the isobutene present in stream C is converted to ATBE (alkyl tert-butyl ether) and a reaction output is obtained, wherein the reaction output is subjected to a product separation in which a residual stream, containing at least alcohol, and a product stream, containing at least the ATBE, are obtained;
e) supplying the residual stream to a first separation unit, wherein in the first separation unit a low-boiler stream, containing at least 1,3-butadiene, 1-butene, 2-butene and isobutane, and a water-containing stream, containing at least alcohol, are obtained;
f) supplying the water-containing stream to a recovery unit, in order to at least partially separate the alcohol off from the water and recycling at least a portion of the alcohol, thus obtained to the reaction unit;
g) supplying the low-boiler stream to a hydrogenation, in order to hydrogenate at least a portion of the 1,3-butadiene present, thus obtaining a hydrogenated low-boiler stream;
h) supplying the hydrogenated low-boiler stream to a second separation unit, wherein in the second separation unit a stream D, containing at least isobutane and 1-butene, and a stream E, containing at least n-butane and 2-butene, are obtained;
i) supplying stream D to a third separation unit, wherein in the third separation unit a crude isobutane stream and a stream of high-purity 1-butene are obtained;
j) supplying the crude isobutane stream to a fourth separation unit, wherein in the fourth separation unit an offgas stream and a stream of high-purity isobutane are obtained.

2. The process according to claim 1, comprising:
supplying the isobutane-depleted stream from b) to a heavy-boiler separation and/or a separation of nitrogen-containing and/or sulfur-containing and/or oxygen-containing impurities, before the isobutane-depleted stream is sent to c).

3. The process according to claim 1, comprising:
effecting the separation at least of a portion of the isobutane-depleted stream in c) via a valve with flow control.

4. The process according to claim 1, comprising:
carrying out the conversion in d) in at least two reaction stages, with at least the last reaction stage being carried out as a reactive distillation.

5. The process according to claim 1, comprising:
washing out, in e), the methanol from the residual stream in an extraction with water or an aqueous solution.

6. The process according to claim 1, comprising:
effecting the hydrogenation of the at least a portion of the 1,3-butadiene in g) in at least two reaction stages, with at least the last reaction stage being carried out in the presence of 0.05 to 100 ppmw of CO.

7. The process according to claim 1, wherein the conversion of the isobutene in d) is above 70%.

8. The process according to claim 1, wherein, as catalyst, an ion exchange resin is used in the conversion of isobutene in d).

9. The process according to claim 1, wherein the high-purity 1-butene obtained in i) has a purity of at least 99%.

10. The process according to claim 1, wherein the high-purity 1-butene obtained in i) contains less than 5000 ppm by mass of isobutene.

11. The process according to claim 1, wherein the high-purity isobutane obtained in j) has a purity of at least 99%.

12. The process according to claim 1, wherein the high-purity 1-butene obtained in i) contains less than 1000 ppm by mass of olefins.

13. The process according to claim 1, wherein the first separation unit and/or the third separation unit and/or the fourth separation unit comprises at least one distillation column.

14. An apparatus for carrying out the process according to claim 1, the apparatus comprising:
an isobutane separation unit comprising at least one distillation column in which at least a portion of the isobutane present in the first C4 hydrocarbon stream A is separated off;
a reaction unit comprising at least one reactor and in which at least a portion of the isobutene present in stream C is converted to MTBE and/or to isobutene dimers;
a first separation unit comprising at least one distillation column and in which a low-boiler stream, containing at least 1,3-butadiene, 1-butene, 2-butene and isobutane, is separated off;
a recovery unit for recovering methanol;
a hydrogenation unit comprising at least one hydrogenation reactor and in which at least a portion of the 1,3-butadiene present in the low-boiler stream is selectively hydrogenated;
a second separation unit comprising at least one distillation column and in which a stream D, containing at least isobutane and 1-butene, is separated off;
a third separation unit comprising at least one distillation column and in which high-purity 1-butene is separated off; and
a fourth separation unit comprising at least one distillation column and in which high-purity isobutane is separated off.
